(19) United States
(12) Patent Application Publication
(10) Pub. No.: US 2009/0162421 A1
(43) Pub. Date: Jun. 25, 2009

(54) DRUGS AS WELL AS THEIR PRODUCTION AND USE IN THE TREATMENT OF PAIN-ASSOCIATED NEUROPATHIES

(75) Inventors: Gerd GEISSLINGER, Bad Soden (DE); Irmgard Tegeder, Frankfurt (DE)

Correspondence Address: FOLEY AND LARDNER LLP;SUITE 500, 3000 K STREET NW, WASHINGTON, DC, 20007, US

(73) Assignee: PAZ Arzneimittel-Entwicklungsgesellschaft mbH

(21) Appl. No.: 12/388,875

(22) Filed: Feb. 19, 2009

Related U.S. Application Data
(63) Continuation of application No. PCT/EP2008/010592, filed on Dec. 12, 2008.

(30) Foreign Application Priority Data
Dec. 21, 2007 (DE) 10 2007 062 139.8

(51) Int. Cl. A61K 9/70; A61K 31/192; A61K 31/381; A61K 31/55; A61P 29/00; A61K 31/485; A61K 9/28; A61K 9/127

(52) U.S. Cl. 424/445; 514/568; 514/438; 514/217; 514/282; 424/474; 424/450

(57) ABSTRACT

The present invention relates to the use of tarenflurbil and/or a pharmaceutically tolerable salt or derivative thereof in enantiomerically-pure and/or essentially enantiomerically-pure form or a form that is enriched with respect to flurbiprofen racemate and/or a racemate of said salt or derivative, for the production of a drug for the treatment of pain-associated neuropathy, pain-associated neuropathy that is simultaneously accompanied by states of nociceptive pain, peripheral and/or predominantly peripheral neuropathic pain or central and/or predominantly central neuropathic pain.

The present invention is related to drugs which contain tarenflurbil (proposed INN name; previous name R-flurbiprofen) in pure form or enriched with respect to the racemate as active ingredient, to the production and use of said drugs in pharmaceutical preparation for systemic or topical application as rapid- or modified-release formulations for the treatment of pain-associated neuropathies or neuropathic pain in mammals, in particular in man.

### BACKGROUND OF THE INVENTION

Neuropathies are diseased conditions of the nerves that can be related to two very different types of disease. One type of disease is the disease due to nerve damage in terms of a somatic disease. The other type of disease is related to the nerve system in terms of an irritable weakness (e.g. nervousness, neurasthenia, neurosis). The latter meaning is to be seen more in a historic context; the term, mental disease, is used more commonly nowadays.

Somatic neuropathies can have various causes, e.g. severing of the nerve due to amputation or accident, blood circulation disorder upon manifestation of arterial occlusive disease or diabetes mellitus, mechanically damaging influences (traumata) acting on the nerve, tumors, metabolic (metabolism-related) disorder or nerve damage due to diabetes mellitus, vitamin deficiency, hepatic or renal disease, infectious diseases such as Herpes zoster, toxic diseases due to alcohol, heavy metals, medications, cyclic hydrocarbons, autoimmune diseases, mainly in the case of Guillain-Barré syndrome, and central nervous disorders or damage affecting the spinal cord or brain.

There are numerous options for the treatment of neuropathy depending on the cause of the disease. Therapeutic algorithms currently in use take into account the therapy of the underlying cause, the pharmacological and non-pharmacological therapy of disease symptoms as well as supportive psychological therapy.

Since the pain conduction system itself is disturbed or damaged in somatic neuropathies, inherent nerve pain, so-called neuropathic pain, occurs often. This type of pain is characterized by burning, lightning-like spontaneous pain, shooting pain attacks, and evoked pain (allodynia, hyperalgesia), but also by paresthesia and hypesthesia.

Neuropathic pain is completely different from nociceptor pain since the causes are different and the etiology is different. In contrast to neuropathic pain, nociceptor pain occurs after tissue damage or inflammation, in which the peripheral and central nerve structures are intact. Therefore, the triggering, conduction, and central processing of the pain impulses by the peripheral and central nervous system, i.e. so-called nociception, is fully functional in nociceptor pain. Nociceptor pain includes, e.g., all chronic inflammation pain, visceral pain, many components of chronic back pain, and most of the components of tumor pain.

Because of the difference in the cause of disease, course of disease, and medication therapy, the Committee for Proprietary Medicinal Products (CPMP) of the European Agency for the Evaluation of Medicinal Products (EMEA) issued different official guidelines for the clinical development of drugs for the treatment of neuropathy-related pain [Guideline on Clinical Medicinal Products intended for the Treatment of Neuropathic Pain, CPMP/EWP/252/03 Rev. 1, 24 Jan. 2007] and nociceptive pain [Note for Guidance on Clinical Investigation of Medicinal Products for Treatment of Nociceptive Pain, CPMP/EWP/612/00, 21 Nov. 2002].

Pain-associated neuropathies impair the quality of life of the patient significantly and are a major health-economy problem. The point prevalence of pain-associated neuropathies in the general population is estimated to be approx. 5% according to a survey in six European countries [McDermott A M, Tölle T R, Rowbotham D J, Schaefer C P, Dukes E M: The burden of neuropathic pain: results from a cross-sectional survey. Eur J Pain 2006; 10(2): 127-135.]. During the “Second International Congress on Neuropathic Pain” of the International Association for the Study of Pain (IASP) held 7-10 Jun. 2007 in Berlin, it was noted that neuropathic pain becomes manifest in 8% of the brain infarction patients, 20% of the diabetics, 28% of the multiple sclerosis patients, approx. 33% of the patient with tumor pain, 37% of the patients with back pain, and 67% of the patients with spinal cord injuries in Germany.

Pain as a consequence of a neuropathy requires a different medicinal therapy than nociceptor pain. It is therefore essential to diagnose the manifestation of neuropathic pain and differentiate this from nociceptor pain, which is more common in medical practice, before initiating any treatment. Several validated procedures are available for safe diagnosis of neuropathic pain [overview in: Baron, R: Diagnostik und Therapie neuropathischer Schmerzen (Detection of neuropathic pain syndromes), Deutsches Ärzteblatt 103, issue 41, 2006, 2720-30]. The diagnostic options range from clinically-oriented diagnoses [Dworkin R H, Backonja M, Rowbotham M C, Allen R R, Argoff C R, Bennett G J et al.: Advances in neuropathic pain: diagnosis, mechanisms, and treatment recommendations. Arch Neurol 2003; 60(11): 1524-34. Cruccu G, Anand P, Attal N, Garcia-Larrea L, Haanpaa M, Jorum E et al.: EFNS guidelines on neuropathic pain assessment. Eur J Neurol 2004; 11 (3): 153-62. Jensen T S, Baron R: Translation of symptoms and signs into mechanisms in neuropathic pain. Pain 2003; 102(1-2):1-8] up to simple questionnaires [Freynhagen R, Baron R, Gockel U, Tölle T R: painDETECT—a new screening questionnaire to identify neuropathic components in patients with back pain. Curr Med Res Opin 2006; 22(10): 1911-20].

In accordance with current therapeutic guidelines [Baron R, Ludwig J, Binder A: Therapie Tabellen Neurologie/Psychiatrie no. 29, May 2006, Neuropathische Schmerzen, Westermayer Verlag, Pentenried], the following drugs are available for pharmacotherapy of neuropathic pain: antidepressants (e.g. amitriptyline, nortriptyline; desipramine, maprotiline, venlafaxine, duloxetine, bupropion), anticonvulsants (e.g. carbamazepine, oxcarbazepine, lamotrigine, gabapentin, pregabalin), opioids (e.g. tramadol, morphine, oxycodone), cannabinoids (e.g. tetrahydro-cannabinol), myotonolytics (e.g. baclofen), and NMDA (═N-Methyl-D-aspartate) antagonists (e.g. dextromethorphan, ketamine, memantine), radical scavengers (e.g. alpha-lipoic acid). Topical drugs to be considered include local analgesics and local anesthetics (e.g. capsaicin, lidocaine, and benzocaine).

Both opioid analgesics and non-opioid analgesics are in use for pharmacotherapy of nociceptive pain. The non-opioid analgesics include the non-steroidal antiinflammatory drugs (NSAIDs) including the COX-2-selective inhibitors which have an analgesic, antiphlogistic, and antipyretic effect. NSAIDs have not been included in the current therapeutic recommendations and official guidelines of EMEA for the treatment of pain-associated neuropathies because they have proven to be ineffective and would therefore only cause side effects. This applies, e.g., to ibuprofen [Baron, R: Diagnostik und Therapie neuropatischer Schmerzen (Detection of neuropathic pain syndromes), Deutsches Ärzteblatt 103, issue 41, 2006, 2720-30. Max, M B et al.: Association of pain relief with drug side effects in postherpetic neuralgia: a single-dose study of clonidine, codeine, ibuprofen, and placebo, Clin Pharmacol Ther. 1988; 43(4), 363-71] as well as diclofenac, indo-methacin and aspirin [Hempenstall, K et al.: Analgesic therapy in postherpetic neuralgia: A quantitative systematic review. PLoS Med. 2005; 2(7), 1-27].

Therefore, according to the current therapeutic recommendations, of the pain medications currently available, only opioid analgesics are well-suited for the treatment of both pain-associated neuropathies and for the treatment of nociceptive pain. All other agents are well-suited only for either the treatment of pain-associated neuropathies or only for the treatment of nociceptive pain due to the difference in the cause of pain and due to their mechanism of action. Accordingly, mixed pain syndromes, consisting of neuropathic and nociceptive pain, must usually be treated individually using the agents that are well-suited for the corresponding type of pain.

Some of the well-known NSAIDs contain an asymmetric carbon atom and therefore form an R-enantiomer and an S-enantiomer. This includes the agent class of the 2-aryl propionic acids, which includes well-known substances such as, e.g., ibuprofen, flurbiprofen, ketoprofen, naproxen and tiaprofenic acid. Both ibuprofen and ketoprofen are used for therapy both in the form of the racemate (50% S-enantiomer, 50% R-enantiomer) and in the form of the pure S-enantiomer, whereby only the S-enantiomer is considered to be effective. Naproxen is used in drugs exclusively in the form of the S-enantiomer. Flurbiprofen and tiaprofenic acid are thus far being used for therapy only in the form of the racemate.

According to the prior art and contrary to previous scientific insight, some 2-aryl propionic acids are now known to be pharmacologically effective not only in the form of the S-enantiomer, but also show desired pharmacological effects, in particular an analgesic effect, in the form of the R-enantiomer, please refer to, e.g., DE 40 28 906, EP 0 607 128, U.S. Pat. No. 5,200,198, and U.S. Pat. No. 5,206,029. These documents relate to nociceptive pain exclusively.

WO 00/13684 describes that some R-(2)-aryl propionic acids, preferably, tarenflurbil, have antinflammatory effects at doses exceeding the effective doses for analgesia, whereby the mechanism of action is stated to be an inhibition of the induction of COX-2 at mRNA level.

EP 1 154 766 claims the use of R-(2) aryl propionic acids, preferably tarenflurbil, for the production of drugs for fighting rheumatic diseases, asthma, shock, inflammatory bowel diseases, radiation damage, arteriosclerosis, and rejection reactions after tissue or organ transplantations, whereby the effect in these diseases is said to be based on the inhibition of transcription factor NF-kappaB activation.

Further applications of R-(2) aryl propionic acids, preferably of tarenflurbil, as chemo-preventative agents for the treatment of neoplastic diseases (colorectal cancer) as well as for the treatment of cystic fibrosis and Alzheimer's disease are described in WO 98/09603.

EP 1 322 305 claims the use of R-(2) aryl propionic acids, preferably of tarenflurbil, for the treatment of chronic-destructive cartilage and articular diseases in the presence of rheumatism.

Ketoprofen has been described to trigger an inhibitory effect on tactile allodynia in rats after spinal application of R-ketoprofen or of mixtures of morphine and S-ketoprofen. This is taken to conclude that spinal application of these substances may be well-suited for the treatment of neuropathic pain [Ossipov M H, Jerussi T P, Ren K, Sun H, Porreca F: Differential effects of spinal (R)-ketoprofen and (S)-ketoprofen against signs of neuropathic pain and tonic nociception: evidence for a novel mechanism of action of (R)-ketoprofen against tactile allodynia. Pain. 2000 August; 87(2):193-9]. The pain model used suggests that not just neuropathic pain was addressed, but rather nociceptive pain or mixed pain consisting of nociceptive and neuropathic pain. The spinal application selected in the animal model is limited in its clinical applicability due to the extensive effort involved. This type of application would only permit the treatment of central neuropathic pain, whereas treatment of peripheral neuropathic pain would not be feasible. The continuous use of this type of application is feasible with extensive technical effort only, e.g. with implantable pumps. A pharmacological effect in other types of applications as are common in the practice of long-term therapy has not been described for this substance. The results of this scientific publication are the basis of U.S. Pat. No. 6,620,851 B2, in which procedures for the treatment of neuropathic pain and other disorders through the use of R(−)-ketoprofen are claimed. Oral types of administration are mentioned, but no evidence of their efficacy is provided. The efficacy of pure, orally-administered R-ketoprofen cannot be demonstrated either in rats or humans, since R-ketoprofen is converted to S-ketoprofen to a significant extent after oral application in both species. As a result, any possibly existing effect of R-ketoprofen cannot be distinguished from the stronger effect of S-ketoprofen. Moreover, bioinversion of R-ketoprofen to S-ketoprofen after oral administration would also lead to the well-known adverse effects of S-ketoprofen and thus might ruin any better tolerability of R-ketoprofen.

The expert cannot assume that the effect found with R-ketoprofen is applicable to other R-aryl propionic acids due to the very different effects and/or effective dosages and, mainly, the strongly different degree of inversion varying from species to species and from aryl propionic acid to aryl propionic acid found in the studies carried out thus far. As such, unlike the S-enantiomers, the R-aryl propionic acids cannot be considered to be one uniform class of agents.

There continues to be a need for therapeutic procedures and/or drugs that are well-suited for the treatment of pain-associated neuropathies.

### SUMMARY OF THE INVENTION

It has been found surprisingly that the application of tarenflurbil in pharmacological models of neuropathic pain in rats leads to a significant reduction of the evoked neuropathic pain.

As expected, S-flurbiprofen, which is very effective against nociceptive pain, did not show this effect. According to current insights, rats are the only established species of laboratory animals that almost not invert tarenflurbil to S-flurbiprofen under in vivo conditions. For this reason, this species is well-suited for selective determination of the effects of tarenflurbil, whereby the effects found are applicable to humans.

For this reason, the present invention relates to a procedure for the treatment of pain-associated neuropathy; of pain-associated neuropathy accompanied by states of nociceptive pain; of peripheral and/or predominantly peripheral neuropathic pain; or of central and/or predominantly central neuropathic pain in a mammal, in particular in man.

In particular, the invention relates to a procedure for the treatment of the following types of neuropathic pain and/or neuropathic pain due to a cause that is selected from the group of the following causes: systemic diseases, e.g. diabetic neuropathy; drug-induced lesions, e.g. neuropathy due to chemotherapy; traumatic syndrome and entrapment syndrome; lesions in nerve roots and posterior ganglia; neuropathies after HIV infections; neuralgia after Herpes infections; nerve root avulsions; cranial nerve lesions; cranial neuralgias, e.g., tri-geminal neuralgia; neuropathic cancer pain; phantom pain; compression of peripheral nerves, neuroplexus and nerve roots; paraneoplastic peripheral neuropathy and ganglionopathy; complications of cancer therapies, e.g. chemotherapy, irradiation, and surgical interventions; complex regional pain syndrome; type I lesions (previously known as sympathetic reflex dystrophy); and type II lesions (corresponding approximately to causalgia).

Moreover, the invention relates to a procedure for the treatment of neuropathic pain due to a cause that is selected from the following group of causes: cerebral lesions that are predominantly thalamic; infarction, e.g. thalamic infarction or brain stem infarction; cerebral tumors or abscesses compressing the thalamus or brain stem; multiple sclerosis; brain operations, e.g. thalamotomy in cases of motoric disorders; spinal cord lesions; spinal cord injuries; spinal cord operations, e.g. anterolateral cordotomy; ischemic lesions; anterior spinal artery syndrome; Wallenberg's syndrome; and syringomyelia.

According to the invention, enantiomerically-pure or essentially enantiomerically-pure tarenflurbil and/or a pharmaceutically tolerable salt or derivative thereof, or tarenflurbil and/or pharmacologically tolerable tarenflurbil derivative or salt that is enriched with respect to flurbiprofen racemate and/or a racemate of said salt or derivative is administered to a mammal, in particular to a human, who is afflicted by said pain-associated neuropathies or neuropathic pain.

Moreover, the present invention relates to the use of tarenflurbil and/or a pharmaceutically tolerable salt or derivative thereof in enantiomerically-pure or essentially enantiomerically-pure form or in a form that is enriched with respect to flurbiprofen racemate and/or a racemate of said salt or derivative, for the production of a drug for the treatment of said pain-associated neuropathies or neuropathic pain in a mammal, in particular in a human.

Moreover, the invention relates to tarenflurbil and/or a pharmaceutically tolerable salt or derivative thereof in enantiomerically-pure or essentially enantiomerically-pure form or in a form that is enriched with respect to flurbiprofen racemate and/or a racemate of said salt or derivative, and drugs and/or drug compositions containing these for use for the treatment of said pain-associated neuropathies or neuropathic pain in a mammal, in particular in a human.

### SHORT DESCRIPTION OF THE FIGURES

FIGS. 1a, 1b, and 1c show the dose-dependent effects of tarenflurbil (=R-flurbiprofen; RF) in the spared nerve injury model (SNI model, graphs on the left) and in the chronic constriction injury model (CCI model; graphs on the right) of peripheral neuropathic pain as compared to the effects of S-flurbiprofen (SF), gabapentin (Gaba) and vehicle (vehicle).

FIG. 2 shows the effects of a treatment, from the first postoperative day, with tarenflurbil (═R flurbiprofen; RF), by administration of a dose of 9 mg/kg twice daily i.p., as compared to vehicle in the spared nerve injury model of peripheral neuropathic pain.

FIG. 3 shows the effects of tarenflurbil (single i.p. dose of 9 mg/kg) as compared to vehicle in untreated rats.

### DETAILED DESCRIPTION OF THE INVENTION

Although NSAIDs are applied broadly in the treatment of nociceptive pain and inflammatory diseases, they have proven ineffective for the treatment of pain-associated neuropathies in the studies that have been carried out thus far and are therefore not included in the therapeutic algorithms for the treatment of pain in association with neuropathies.

In the context of the present invention, tarenflurbil, the R-enantiomer of the NSAID, flurbiprofen, which is commercially available in the form of the racemate, was surprisingly found to be effective in terms of pain relief in the treatment of pain-associated neuropathies. The effect of tarenflurbil in pain-associated neuropathies is selective and is not elicited by the S-enantiomer of flurbiprofen.

The pharmacological investigations that have been carried out in the context of the present invention show the dose-dependent effect of tarenflurbil in pharmacologically accepted models of pain-associated neuropathies. If dosed sufficiently, the efficacy is in the range of the efficacy of 25 mg/kg body weight gabapentin as reference substance. S-flurbiprofen does not show a significant effect in these models.

Tarenflurbil (and/or a pharmaceutically tolerable salt or derivative thereof)-containing drugs can therefore be used in the scope of the invention in all forms of pain-associated neuropathies or neuropathic pain described above as mono-therapy or in combination with other drugs or treatment procedures. The dosing should follow the current therapeutic guidelines for the known agents. This means that the dosing should be designed individually, depending on both the efficacy and adverse effects.

In the context of the treatment of pain-associated neuropathies and/or neuropathic pain, efficacy is understood to be a reduction of pain by at least 30 to 50%. This should preferably be evaluated only after a treatment period of two to four weeks on high daily doses. After this period, the dose can be adjusted upwards or downwards or a combination therapy including the known agents can be initiated.

Since the adverse drug effects of flurbiprofen racemate are essentially caused by the S-enantiomer, it is particularly advantageous in the scope of the invention to use and/or administer tarenflurbil and/or its pharmaceutically tolerable salts or derivatives in enantiomerically-pure or essentially enantiomerically-pure form. Forms of tarenflurbil and/or its pharmaceutically tolerable salts or derivatives, in which tarenflurbil and/or the pharmaceutically tolerable salt or derivative is enriched with respect to the corresponding racemic form consisting of 50% tarenflurbil or pharmaceutically tolerable salt or derivative and 50% S-flurbiprofen and/or pharmaceutically tolerable salt or derivative thereof are also well-suited for this purpose. The higher the enantiomeric excess of tarenflurbil and/or pharmaceutically tolerable salt or derivative with respect to the corresponding S-enantiomer, the higher the drugs made therefrom can be dosed, e.g., in order to attain the desired analgesic effect in the treatment of pain-associated neuropathies in combination with an incidence and severity of the adverse drug effects as low as possible.

Particularly well-suited for this purpose are forms of tarenflurbil and/or forms of the pharmaceutically tolerable salt or derivative thereof, in which tarenflurbil and/or its pharmaceutically tolerable salt or derivative is present at a molar ratio of larger than or equal to 60:40 with respect to S-flurbiprofen and/or the corresponding pharmaceutically tolerable salt or derivative of S-flurbiprofen. It is particularly preferred for the molar ratio to be larger than or equal to 95:5, whereby a molar ratio from 95:5 is “essentially enantiomerically-pure” in the scope of the invention. It is even more particularly preferred for the molar ratio to be larger than or equal to 98:2, ≧99.5:0.5 or >99.9:0.1, whereby a molar ratio from 98:2 is “enantiomerically-pure” in the scope of the invention.

The drugs and drug compositions made in accordance with the use according to the invention and/or the drugs and drug compositions for use according to the invention preferably contain tarenflurbil and/or its pharmaceutically tolerable salt or derivative in said well-suited forms of enantiomeric purity or enrichment.

If reasonable on technological grounds during the production of the tarenflurbil agent, pure tarenflurbil and/or its pharmaceutically tolerable salt or derivative, in which no S-flurbiprofen and/or corresponding S-flurbiprofen salt or derivative can be detected with modern analytical methods, should be used in the procedures according to the invention and/or for the production of the drugs and/or drug compositions according to the invention.

Unlike most of the other R—NSAIDs, tarenflurbil shows no or no more than extremely minor inversion of the R-form to the S-form under in vivo conditions when applying it in humans. For this reason, even after the application of high therapeutic doses, there is no risk of toxic concentrations of S-flurbiprofen occurring in the human organism due to the inversion of tarenflurbil to S-flurbiprofen. Due to the high enantiomeric stability of tarenflurbil under in vivo conditions in man, drugs should be produced to contain the agent as enantiomerically-pure as possible in order to obtain a particularly favorable benefit-risk relationship in the treatment of pain-associated neuropathies or neuropathic pain according to the invention. Enantiomerically-pure or essentially enantiomerically-pure tarenflurbil is available in a form produced by several commercial agent producers in compliance with the international Good Manufacturing Practices (GMP) for pharmaceutical agents.

All specifications of well-suited daily doses, single doses, and agent concentrations made in the present description of the invention and the claims refer to tarenflurbil and/or its corresponding pharmaceutically tolerable salt or derivative including the fraction of S-flurbiprofen and/or S-enantiomer of the corresponding pharmaceutically tolerable salt or derivative that may also be present in addition in accordance with the degree of enantiomeric purity or enrichment of the form of tarenflurbil or its pharmaceutically tolerable salt or derivative that is used.

Because of the low toxicity, the administered daily dose can be adjusted widely to the individual situation of the patient. Upon systemic application, i.e. if the administered dose is to be made available by means of the blood circulation in the organism, the daily dose should be at least 1 mg/kg body weight and can be increased up to 50 mg/kg or higher. A preferred dosage for systemic application is a daily dose of 2 to 30 mg/kg body weight, 3 to 25 mg/kg body weight is particularly preferred, 5 to 20 mg/kg body weight is even more particularly preferred, and 10 to 20 mg/kg body weight is most preferred.

Depending on the release kinetics of the pharmaceutical preparation and the form of application, the daily dose should be split into one to five, preferably one to four units such that an application is made once to five times, preferably four times, daily.

All known application routes in which the known pharmaceutical formulations can be used, such as oral, peroral, intramuscular, intravenous, intraperitoneal, buccal, sublingual, nasal, transdermal, by inhalation, and rectal, can be used for application.

Oral or rectal formulations that are administered in the form of a solid preferably contain as single dose form 30 mg to 1800 mg, more preferably 50 mg to 1200 mg, particularly preferably 100-1000 mg, and more particularly preferably 200-800 mg tarenflurbil and/or a pharmaceutically tolerable salt or derivative thereof. For suppositories, single doses are split similarly to solid oral formulations.

Solutions or suspensions for parenteral administration preferably contain the entire daily dose in a single dose form, but can also be prepared as lower-dosed single dose forms for multiple daily application according to therapeutic need.

In the case of oral drink preparations, the entire daily dose can be taken at once, even if it exceeds 1800 mg. Solutions or suspensions for oral application can contain the requisite quantity for a typical therapeutic period, such as, e.g., one or several weeks or one or several months, whereby in this case a daily dose or a single dose is obtained by measuring an aliquot.

Aside from systemic application, tarenflurbil and/or a pharmaceutically tolerable salt or derivative thereof can be used for local therapy of pain-associated neuropathies or neuropathic pain. For this purpose, a pharmaceutical formulation for topical application is applied to the skin on the site of the body afflicted by the disease. All pharmaceutical formulations known for topical agents are well-suited for this application route, such as, e.g., ointments, cremes, emulsions, gels, patches.

Formulations with concentrations of tarenflurbil or its pharmaceutically tolerable salt or derivative of 0.5 g/100 g to 25 g/100 g of preparation, preferably of 1 g/100 g to 20 g/100 g of preparation, particularly preferably of 1 g/100 g to 15 g/100 g of preparation, more particularly preferably of 1.5 g/00 g to 10 g/100 g of preparation, and most preferably of 5 g/100 g to 10 g/100 g of preparation are well-suited for topical applications.

The pharmaceutical formulation of the drugs and/or drug compositions according to the invention can be produced in any form that is desired and known in pharmaceutical technology. This includes, e.g., tablets, capsules, coated tablets, granulates, non-sterile solutions and suspensions for oral application, nanopellets, sterile solutions and suspensions for parenteral administration, suppositories, aerosols, ointments, cremes, emulsions, and liposome preparations.

The oral pharmaceutical preparations and preparations for intramuscular or intraperitoneal application according to the invention can be produced and used therapeutically in the form of both rapid-release and modified-release formulations. In particular in the case of the oral preparations, it is advisable to provide pharmaceutical preparations with delayed release in order to reduce the frequency of intake, for improving the patient compliance, and for improving the tolerability. The release can be controlled continuously or in pulses via several individual releases that are staggered over time. In order to reduce the frequency of application of formulations for intramuscular or intraperitoneal application, the preparations for delayed agent release that are known in pharmaceutical technology, such as, e.g., crystal suspensions and biodegradable excipients, can be used.

According to a preferred embodiment of the invention, tarenflurbil or its pharmaceutically tolerable salt or derivative is applied and/or administered in the procedures, uses, and drugs and/or drug compositions according to the invention over an extended period of time, preferably over several weeks or several months, e.g. over more than 3 months, more than 6 months or more than 12 months.

Aside from tarenflurbil and/or its pharmaceutically tolerable salt or derivative, drugs and/or drug compositions according to the invention can comprise other agents as agent as well as one or several pharmaceutically tolerable excipient(s).

The term pharmaceutically tolerable derivatives refers to derivatives made from tarenflurbil and pharmaceutically tolerable inorganic or organic bases for salt formation, alcoholic and phenolic compounds for ester formation or amines for amide formation. This includes, in particular, metal salts, e.g. containing aluminum, calcium, lithium, magnesium, potassium, sodium, and zinc or organic salts, e.g. containing lysine, N,N′-dibenzylethylene diamine, choline, diethanolamine, ethylene diamine, meglumine, trometamine, arginine, and alkylamines with 1 to 6 C-atoms, or esters, e.g. containing aliphatic or isoaliphatic alcohols with 1 to 8 C-atoms. It is also included in the scope of the invention to use tarenflurbil in the form of the free acid.

According to the invention, tarenflurbil and/or the pharmaceutically tolerable salt or derivative thereof can be used advantageously in free or fixed combination with other agents that are well-suited for the treatment of pain-associated neuropathies or neuropathic pain.

For medicinal combination therapy of pain-associated neuropathies or neuropathic pain, tarenflurbil and/or a pharmaceutically tolerable salt or derivative thereof can be used in fixed combinations with other substances that are effective in this indication. For systemic combination therapy, substances from the agent groups of the antidepressants, anticonvulsants, opioids, cannabinoids, and myotonolytics are preferable. For topical combination therapy, combinations of tarenflurbil and topical analgesics or local anesthetics can be used. In the production of drugs containing fixed combinations of tarenflurbil and/or a pharmaceutically tolerable salt or derivative thereof and other agents, the doses are selected such that tarenflurbil (and/or the pharmaceutically tolerable salt or derivative thereof that is used) is used at the doses described above and the combination agent is used at the common therapeutic dose of the corresponding mono-agent.

According to the invention, tarenflurbil is therefore well-suited not only for the therapy of pain-associated neuropathies, but also for the treatment of pain that is composed of pain-associated neuropathies and nociceptor pain due to the manifest damage. Mixed pain of this type occurs when pain-associated tissue traumata and pain-associated peripheral or central nerve damage occur in close proximity in the body. Especially this type of pain is often inadequately treated in the practice of medicine, since the known non-opioid analgesics are effective only in the case of nociceptor pain, but not in pain-associated neuropathies, whereas most of the agents used for the treatment of pain-associated neuropathies are effective only in this type of pain, but not against nociceptor pain. Tarenflurbil and/or a pharmaceutically tolerable salt or derivative is effective against mixed pain simultaneously via the known pain-relieving effect against nociceptor pain as well as via the effect against pain-associated neuropathies that has been found according to the invention. Tarenflurbil and its pharmaceutically tolerable salts and derivatives therefore have a known nociceptive pain potential and a previously unknown neuropathic pain potential similar to the opioid agents, but without their well-known addictive potential and respiratory depressive potential. This combination of pain has never before been described for any other non-opioid substance and provides therefore a major, previously unknown, therapeutic benefit. Due to its excellent tolerability, tarenflurbil (and/or its pharmaceutically tolerable salts and derivatives) can be used against mixed pain at high doses from the very beginning. A comparably broad action spectrum against pain-associated neuropathies and nociceptor pain is present, at best, in the opioid analgesics, but these should only be used if strongly indicated because of their adverse effect profile. Back pain and tumor pain are commonly occurring types of mixed pain for the treatment of which tarenflurbil (and/or its pharmaceutically tolerable salts and derivatives) would be a preferable drug independent of a differential diagnosis.

All agents that are used for the therapy of pain-associated neuropathies, such as, e.g., antidepressants (amitriptyline, nortriptyline; desipramine, maprotiline, venlafaxine, duloxetine, bupropion), anticonvulsants (carbamazepine, oxcarbazepine, lamotrigine, gabapentin, pregabalin), opioids (tramadol, morphine, oxycodon), cannabinoids (tetrahydro-cannabinol), myotonolytics (baclofen), NMDA antagonists (dextromethorphane, ketamine, memantine) or radical scavengers (alpha-lipoic acid), are well-suited as further agents that can be produced in combination with tarenflurbil (and/or its pharmaceutically tolerable salts or derivatives), e.g., as drugs for systemic application, and used for medical purposes as fixed combination. For drugs for topical application, local anesthetics (e.g. capsaicin, lidocaine, and benzocaine) can be considered as agents for combination with tarenflurbil and/or its pharmaceutically tolerable salts or derivatives.

According to the invention, pharmaceutically tolerable excipients are to be understood to be carrier substances, such as starch, sugar, microcrystalline cellulose, diluents, granulation aids, lubricants, binding agents, tablet disintegrants, and alike, depending on the formulation. Particularly advantageous pharmaceutical formulations for tarenflurbil-containing drugs are described in EP 0 607 128, EP 0 641 200, and EP 0 615 440.

The examples described in the following are meant to illustrate the invention in more detail without limiting it to the actual embodiments described. Unless specified otherwise, all part and % specifications in the scope of the present invention refer to the weight and/or the total weight of the composition/mixture.

### Example 1

The Chronic Constriction Injury model (CCI model) [Bennet G J, Xie Y K: A peripheral mononeuropathy in rat that produces disorders of pain sensation like those seen in man. Pain 1988, 33:87-107] and the Spared Nerve Injury model (SNI model) [Decosterd I, Woolf C J: Spared Nerve injury: an animal model of persistent peripheral neuropathic pain. Pain 2000, 87:149-158] were used as animal models that show essential elements of the clinical pain syndromes in the presence of neuropathies. In both models, the animals sustain defined nerve damage by surgical means. The pain behavior of the animals can be used to quantitatively measure the efficacy of substances in pain-associated neuropathies. The investigations were carried out on rats according to the published models. In rats, it is possible to measure the effect of tarenflurbil exclusively, since they, like man, basically do not invert tarenflurbil to S-flurbiprofen. The same is not true of most other species of laboratory animals.

In a first series of experiments, the animals sustained a nerve lesion by surgical means according to the SNI model and according to the CCI model. From day 10 to day 21 postoperatively, groups of 12 animals each received intraperitoneal administrations of the test substances twice daily. In tests according to the SNI model, in each application, 2.5, 4.5 or 9 mg tarenflurbil (referred to as RF2.5, RF4.5, and RF9), 4.5 mg S-flurbiprofen (SF4.5), pure vehicle (vehicle—as control) or 25 mg gabapentin (Gaba25—as reference substance), each per kg of body weight, were administered. The behavioral parameters that are typical of pain-associated neuropathies were measured, namely mechanical allodynia using Frey's aestesiometer, cold allodynia using the acetone test, and cold hyperalgesia using the cold plate at 2° C. In tests according to the CCI model, 4.5 or 9 mg/kg body weight of tarenflurbil (RF4.5 and RF9), 9 mg/kg body weight S-flurbiprofen (SF9) or vehicle (vehicle) were administered per group. As before, mechanical/static allodynia, cold allodynia and cold hyperalgesia were measured using Frey's hair test, the acetone test, and the cold plate at 2° C., respectively.

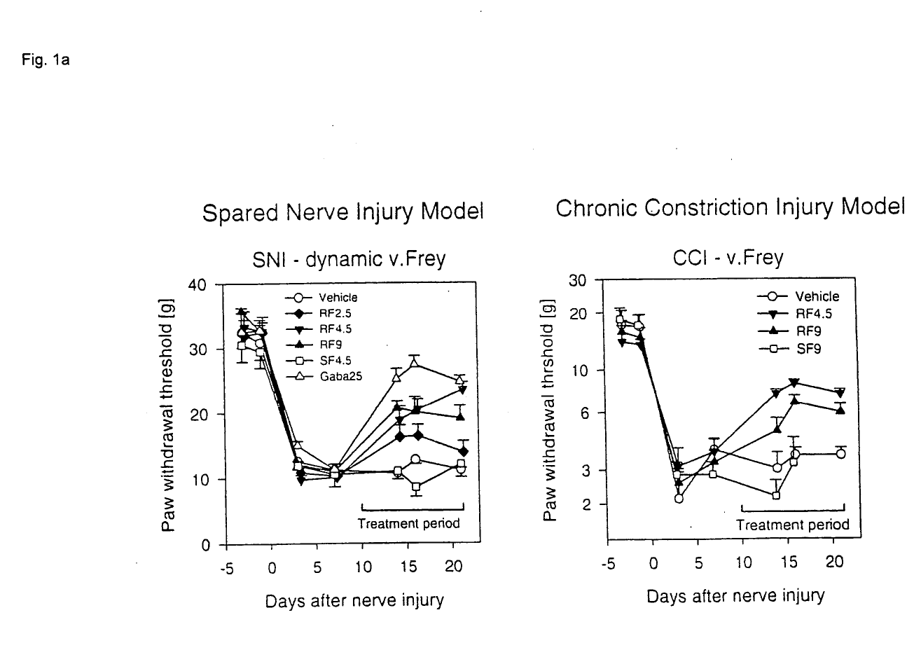

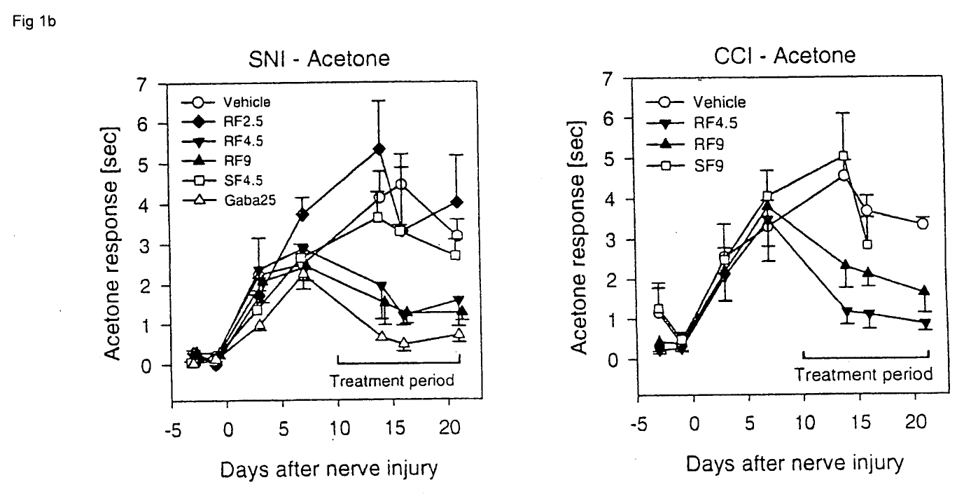

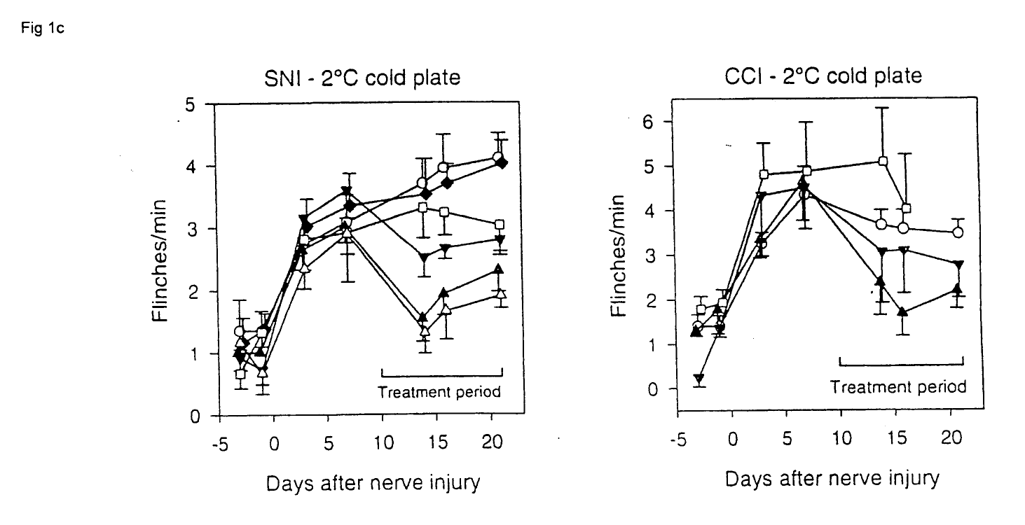

The results of the series' of experiments are shown in FIGS. 1a, 1b, and 1c. A significant reduction of allodynia was attained with 4.5 and 9 mg/kg tarenflurbil twice daily. S-flurbiprofen showed no significant effect. No testing of S-flurbiprofen doses in excess of 4.5 mg/kg twice daily was feasible due to the manifestation of gastrointestinal bleeding in some animals starting after approx. 1 week of treatment. Gabapentin used in neuropathies and here as a positive control showed no significant difference from tarenflurbil.

### Example 2

In another series of experiments, animals having sustained nerve damage according to the SNI model were treated twice daily with intraperitoneal doses of 9 mg/kg body weight tarenflurbil or vehicle from the first postoperative day and for a period of two weeks. Both the mechanical dynamic allodynia (on the ipsilateral and on the contralateral side) and the cold allodynia (ipsilateral only) were measured by means of Frey's aesthesiometer and the cold plate at 10° C., respectively.

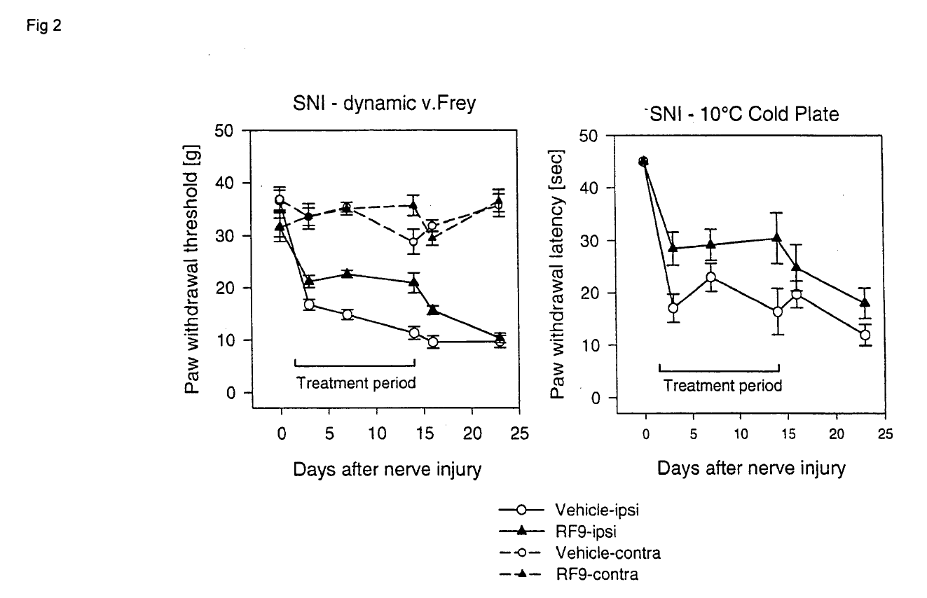

The results of this series of experiments are summarized in FIG. 2. Tarenflurbil (RF9-ipsi) shows a significantly different effect from vehicle (vehicle-ipsi) on the ipsilateral side with regard to mechanical allodynia and cold allodynia during the entire treatment period of two weeks. On the contralateral side, there is no difference between tarenflurbil (RF9-contra) and vehicle (vehicle-contra). After completion of treatment, the pain-relieving effect decreases slowly in the group treated with tarenflurbil and reaches the pain intensity of the vehicle group eight days after the end of treatment.

### Example 3

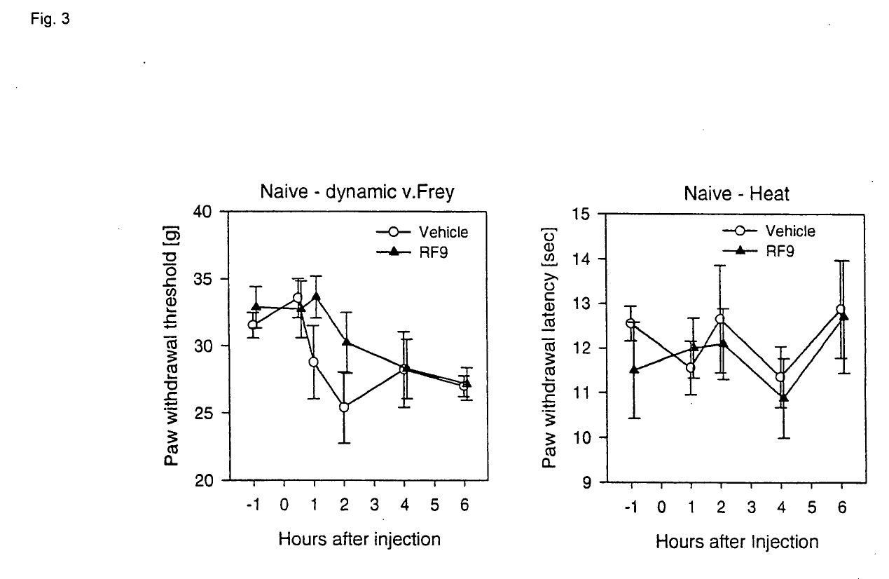

For qualification of the series' of experiments mentioned above, a single dose of 9 mg/kg body weight tarenflurbil (RF9) or vehicle (vehicle) was applied intraperitoneally in another series of experiments on rats that had not sustained damage and the mechanical allodynia and the heat sensitivity were measured for up to 6 hours after the application using Frey's aesthesiometer and the Hargreaves model, respectively. The results shown in FIG. 3 evidence no significant differences between the groups treated with tarenflurbil versus vehicle. This means that the effects measured for tarenflurbil in Examples 1 and 2 are exclusively related to the effect in pain-associated neuropathies.

1. Use of tarenflurbil and/or a pharmaceutically tolerable salt or derivative thereof in a form that is enriched with respect to flurbiprofen racemate and/or a racemate of said salt or derivative, for the production of a drug for the treatment of
a) pain-associated neuropathy
b) pain-associated neuropathy that is simultaneously accompanied by states of nociceptive pain
c) peripheral and/or predominantly peripheral neuropathic pain; or
d) central and/or predominantly central neuropathic pain.

2. Use according to claim 1, whereby the predominantly peripheral neuropathic pain is of a type that is selected from the following types of neuropathic pain and/or has a cause that is selected from the group of the following causes:
systemic diseases, e.g. diabetic neuropathy;
drug-induced lesions, e.g. neuropathy due to chemotherapy;
traumatic syndrome and entrapment syndrome;
lesions in nerve roots and posterior ganglia;
neuropathies after HIV infections;
neuralgia after Herpes infections;
nerve root avulsions;
cranial nerve lesions;
cranial neuralgias, e.g., tri-geminal neuralgia;
neuropathic cancer pain;
phantom pain;
compression of peripheral nerves, neuroplexus and nerve roots;
paraneoplastic peripheral neuropathy and ganglionopathy;
complications of cancer therapies, e.g. chemotherapy, irradiation, and surgical interventions;
complex regional pain syndrome;
type I lesions (previously known as sympathetic reflex dystrophy); and
type II lesions (corresponding approximately to causalgia).

3. Use according to claim 1, whereby the predominantly central neuropathic pain is of a type that has a cause that is selected from the following group of causes:
cerebral lesions that are predominantly thalamic;
infarction, e.g. thalamic infarction or brain stem infarction;
cerebral tumors or abscesses compressing the thalamus or brain stem;
multiple sclerosis;
brain operations, e.g. thalamotomy in cases of motoric disorders;
spinal cord lesions;
spinal cord injuries;
spinal cord operations, e.g. anterolateral cordotomy;
ischemic lesions;
anterior spinal artery syndrome;
Wallenberg's syndrome; and
syringomyelia.

4. Use according to claim 1, whereby tarenflurbil and/or its pharmaceutically tolerable salt or derivative is present at a molar ratio of larger than or equal to 60:40 with respect to S-flurbiprofen and/or the corresponding pharmaceutically tolerable salt or derivative of S-flurbiprofen.

5. Use according to claim 4, whereby tarenflurbil and/or its pharmaceutically tolerable salt or derivative is essentially enantiomerically-pure, i.e. the molar ratio of tarenflurbil and/or its pharmaceutically tolerable salt or derivative and S-flurbiprofen and/or the pharmaceutically tolerable salt or derivative thereof is larger than or equal to 95:5.

6. Use according to claim 5, whereby tarenflurbil is enantiomerically-pure, i.e. the molar ratio is larger than or equal to 98:2.

7. Use according to claim 6, whereby the molar ratio is larger than or equal to 99.5:0.5.

8. Use according to claim 7, whereby the molar ratio is larger than or equal to 99.9:0.1.

9. Use according to claim 1, whereby tarenflurbil and S-flurbiprofen, if present, are present as the free acid, as salt with inorganic or organic salt-forming agents, as complex with inorganic or organic complex-forming agents, as acid ester or as acid amide.

10. Use according to claim 1, whereby, for systemic application, tarenflurbil and/or its pharmaceutically tolerable salt or derivative is used at a daily dose of at least 1 mg/kg body weight to 50 mg/kg body weight or higher, preferably at daily doses of 2 mg/kg to 30 mg/kg body weight, particularly preferably of 3 to 25 mg/kg body weight, more particularly preferably of 5 to 20 mg/kg body weight, and most preferably of 10 to 20 mg/kg body weight.

11. Use according to claim 1, whereby the daily dose is administered as a single dose or in several single doses.

12. Use according to claim 1, whereby, for oral or rectal formulations to be applied in a single dose, single dose forms with an agent content of 30 mg to 1800 mg, preferably with an agent content of 50 mg to 1200 mg, particularly preferably of 100 to 1000 mg, and more particularly preferably of 200 to 800 mg of the agent, and for potable, oral forms as well as forms for injection, single dose forms of minimally 30 mg up to the maximal daily dose are produced and/or administered.

13. Use according to claim 1, whereby the drug is applied for an extended period of time, preferably over several weeks or months.

14. Use according to claim 1, whereby tarenflurbil and/or its pharmaceutically tolerable salt or derivative is combined with one or more agents, at the common therapeutic dose, that are well-suited for systematic treatment of neuropathic pain, e.g. antidepressants (e.g. amitriptyline, nortriptyline; desipramine, maprotiline, venlafaxine, duloxetine, bupropion), anticonvulsants (e.g. carbamazepine, oxcarbazepine, lamotrigine, gabapentin, pregabalin), opioids (e.g. tramadol, morphine, oxycodone), cannabinoids (e.g. tetrahydro-cannabinol), myotonolytics (e.g. baclofen), and NMDA antagonists (e.g. dextromethorphan, ketamine, memantine), radical scavengers (e.g. alpha-lipoic acid).

15. Use according to claim 1, whereby the agent(s) is/are rapidly-released or modified-released from the drugs, e.g. delayed or in pulses.

16. Use according to claim 1, whereby systemic application is performed by the oral, peroral, intramuscular, intravenous, intraperitoneal, buccal, nasal, transdermal, inhalative, and rectal route.

17. Use according to claim 1, whereby the application is by the oral route and the drug is provided in the form of tablets, capsules, coated tablets, granulate, a non-sterile solution or a suspension.

18. Use according to claim 1, whereby the application is by the parenteral route and the drug is provided in the form of a sterile solution or suspension.

19. Use according to claim 1, whereby the application is by the rectal route and the drug is provided in the form of suppositories.

20. Use according to claim 1, whereby the application is by the oral or nasal route and the drug is provided in the form of an aerosol.

21. Use according to claim 1, whereby the application is by the topical route and the agent concentration for local application in the form of topical agents is less than 0.5 g/100 g to 25 g/100 g of preparation or higher, preferably 1 g/100 g to 20 g/100 g of preparation, particularly preferably 1 g/100 g to 15 g/100 g of preparation, more particularly preferably 1.5 g/100 g to 10 g/100 g of preparation, and most preferably 5 g/100 g to 10 g/100 g of preparation.

22. Use according to claim 21, whereby tarenflurbil and/or its pharmaceutically tolerable salt or derivative is combined with one or more agent(s), at the common therapeutic concentration, that is/are well-suited for topical treatment of pain-associated neuropathies, e.g. capsaicin, lidocaine or benzocaine.

23. Use according to claim 21, whereby the drug is provided in the form of a formulation that is well-suited for topical application to the skin, e.g. an ointment, a crème, a gel, a solution, a liposome preparation, a patch or a coated dressing.

24. Method for the treatment of pain-associated neuropathy; pain-associated neuropathy that is simultaneously accompanied by states of nociceptive pain; peripheral and/or predominantly peripheral neuropathic pain; or central and/or predominantly central neuropathic pain,
comprising the steps: Identification of a patient in need of treatment and administration of an effective dose of tarenflurbil and/or a pharmaceutically tolerable salt or derivative thereof in a form that is enriched with respect to flurbiprofen racemate and/or a racemate of said salt or derivative.

25. Method according to claim 24, whereby the patient is a human.

26. Method according to claim 24, whereby the neuropathy and/or the type of pain is selected from those that have a cause that is selected from the group of the following causes: systemic diseases, e.g. diabetic neuropathy; drug-induced lesions, e.g. neuropathy due to chemotherapy; traumatic syndrome and entrapment syndrome; lesions in nerve roots and posterior ganglia; neuropathies after HIV infections; neuralgia after Herpes infections; nerve root avulsions; cranial nerve lesions; cranial neuralgias, e.g., tri-geminal neuralgia; neuropathic cancer pain; phantom pain; compression of peripheral nerves, neuroplexus and nerve roots; paraneoplastic peripheral neuropathy and ganglionopathy; complications of cancer therapies, e.g. chemotherapy, irradiation, and surgical interventions; complex regional pain syndrome; type I lesions (previously known as sympathetic reflex dystrophy); and type II lesions (corresponding approximately to causalgia); cerebral lesions that are predominantly thalamic; infarction, e.g. thalamic infarction or brain stem infarction; cerebral tumors or abscesses compressing the thalamus or brain stem; multiple sclerosis; brain operations, e.g. thalamotomy in cases of motoric disorders; spinal cord lesions; spinal cord injuries; spinal cord operations, e.g. anterolateral cordotomy; ischemic lesions; anterior spinal artery syndrome; Wallenberg's syndrome; and syringomyelia.
